(19) United States
(12) Patent Application Publication
(10) Pub. No.: US 2021/0169832 A1
(43) Pub. Date: Jun. 10, 2021

(54) METHOD OF TREATING TEMPOROMANDIBULAR JOINT DISORDERS

(71) Applicant: TMJ Pharmaceuticals, LLC, Shorewood, IL (US)

(72) Inventors: Alexander Grabavoy, Shorewood, IL (US); Carl Knutsen, Shorewood, IL (US)

(73) Assignee: TMJ Pharmaceuticals, LLC, Shorewood, IL (US)

(21) Appl. No.: 16/949,603

(22) Filed: Nov. 5, 2020

Related U.S. Application Data
(63) Continuation of application No. 15/967,817, filed on May 1, 2018, now abandoned.
(60) Provisional application No. 62/500,349, filed on May 2, 2017.

(51) Int. Cl. A61K 31/192; A61K 31/135; A61K 9/00; A61K 45/06; A61K 31/167; A61K 9/06; A61K 9/107

(52) U.S. Cl. CPC A61K 31/192; A61K 31/135; A61K 9/0014; A61K 9/107; A61K 31/167; A61K 9/006; A61K 9/06; A61K 45/06

(57) ABSTRACT

The present disclosure generally relates to methods for treating temporomandibular joint disorders. More particularly, the disclosure pertains to topical compositions and methods of applying the topical compositions to reduce pain and symptoms associated with temporomandibular joint disorders. The method may include applying an external composition to a head or neck area and applying an intraoral composition to an intraoral area. The external composition and the intraoral composition may include an anti-inflammatory and a muscle relaxant. The disclosed compositions and methods alleviated facial pain, preauricular pain, jaw pain, neck pain, upper-back pain, and headache.

### CROSS-REFERENCES TO RELATED APPLICATIONS

This application is a continuation application of U.S. patent application Ser. No. 15/967,817, filed May 1, 2018, which claims benefit under 35 U.S.C. § 119(e) of U.S. Provisional Application No. 62/500,349, filed May 2, 2017, both of which are incorporated herein by reference in their entirety.

### BACKGROUND

### 1. Field of the Invention

The present disclosure generally relates to methods for treating temporomandibular joint disorders. More particularly, the disclosure pertains to topical compositions and methods of applying the topical compositions to reduce pain and symptoms associated with temporomandibular disorders.

### 2. Description of the Related Art

Temporomandibular joint disorders (TMJD) encompass ailments associated with the temporomandibular joint (TMJ), muscles of mastication, and secondary support of musculature. Patients with TMJD report symptoms such as limitations on opening or noise during jaw movement and pain. The pain can be localized to the TMJ or patients may experience pain in the neck and shoulders. Other symptoms may include jaw pain, headaches, dizziness, earaches, tinnitus, facial pain, jaw locking, and pain on mastication.

Treatments for TMJD may include behavioral therapy, jaw exercises, occlusal appliances, postural training, and pharmacological agents. Pharmacological agents can be effective in alleviating many of the symptoms associated with TMJD.

### BRIEF SUMMARY

In some embodiments, a method is disclosed for treating a temporomandibular disorder or symptoms thereof. The method may include applying an external composition to a head or neck area and applying an intraoral composition to an intraoral area. The external composition and the intraoral composition may include an anti-inflammatory and a muscle relaxant.

In some embodiments, a method is disclosed for treating pain. The method may include contacting skin with an external composition and contacting mucosal tissue in an oral cavity with an intraoral composition. The external composition and the intraoral composition may include an anti-inflammatory and a muscle relaxant.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### DETAILED DESCRIPTION

Various embodiments are described below. The relationship and functioning of the various elements of the embodiments may better be understood by reference to the following detailed description. However, embodiments are not limited to those illustrated below. In certain instances details may have been omitted that are not necessary for an understanding of embodiments disclosed herein.

The compositions and methods disclosed herein have demonstrated surprising and unexpected results in treating TMJD. The disclosed compositions and methods can alleviate facial pain, preauricular pain, jaw pain, neck pain, upper-back pain, and headache.

In some embodiments, a method is disclosed for treating a TMJD or symptoms thereof. The method may include applying an external composition to a head or neck area and applying an intraoral composition to an intraoral area. The external composition and the intraoral composition may include an anti-inflammatory and a muscle relaxant.

In some embodiments, the anti-inflammatory may be a non-steroidal anti-inflammatory drug. The anti-inflammatory in the external composition and the intraoral composition may include but are not limited to celecoxib, diclofenac, diflunisal, etodolac, ibuprofen, indomethacin, ketoprofen, ketorolac, meloxicam, nabumetone, naproxen, oxaprozin, piroxicam, salsalate, sulindac, tolmetin, or any combination thereof. In some embodiments, the anti-inflammatory in the external and intraoral composition may be different or may be selected independently form the list above, for example the external composition may include ketoprofen and the intraoral composition may include naproxen.

In some embodiments, the anti-inflammatory may be ketoprofen. In some embodiments, the external composition may include ketoprofen. In some embodiments, the intraoral composition may include ketoprofen.

In some embodiments, the muscle relaxant in the external composition and the intraoral composition may include but are not limited to carisoprodol, chlorzoxazone, cyclobenzaprine, metaxalone, methocarbamol, orphenadrine, or any combination thereof. In some embodiments, the muscle relaxant in the external and intraoral composition may be different or may be selected independently form the list above.

In some embodiments, the muscle relaxant may be cyclobenzaprine. In some embodiments, the external composition may include cyclobenzaprine and the intraoral composition may include cyclobenzaprine.

In some embodiments, the intraoral composition or the external composition may include an anticonvulsant. The anticonvulsant may include but is not limited to carbamazepine, clonazepam, diazepam, ethosuximide, gabapentin, metharbital, phensuximide, pregabalin, primidone, tiagabine, topiramate, or any combination thereof. In some embodiments, the anticonvulsant may be gabapentin. In some embodiments, the anticonvulsant may be topiramate.

In certain embodiments, the external composition may include an anesthetic. The anesthetic may include but is not limited to benzocaine, lidocaine, pramoxine, prilocaine, or any combination thereof.

In some embodiments, the anesthetic may be lidocaine, prilocaine, or any combination thereof. In some embodiments, the anesthetic may be lidocaine.

In other embodiments, the external composition may include a carrier. The carrier used in the external composition may include an oil-in-water emulsion. In some embodiments, the carrier may include propylene glycol, ethyl alcohol, or a mixture thereof. A suitable carrier may be a gel, ointment, or cream that allows the drugs in the composition to penetrate the lipid layers of the skin. For example, the carrier may be Lipoderm or VersaPro™ Cream Base sold by MEDISCA®.

In some embodiments, the intraoral composition may include a plasticized ointment. The plasticized ointment facilitates penetration of the squamous cell and basal layers of the mouth.

In some embodiments, the intraoral composition or the external composition may consist of an anti-inflammatory, a muscle relaxant, and a carrier. In some embodiments, the external composition consists of an anti-inflammatory, a muscle relaxant, an anesthetic, and a carrier. In some embodiments, the intraoral composition may consist of ketoprofen, cyclobenzaprine, and a carrier. In some embodiments, the external composition may consist of ketoprofen, cyclobenzaprine, and a carrier. In some embodiments, the external composition may consist of ketoprofen, cyclobenzaprine, lidocaine, and a carrier.

In some embodiments, the intraoral composition and the external composition may include about 5% to about 15% by weight of an anti-inflammatory and about 0.5% to about 2.5% by weight of a muscle relaxant. In some embodiments, the intraoral composition and the external composition may include about 5% to about 15% by weight of ketoprofen and about 0.5% to about 2.5% by weight of cyclobenzaprine.

In some embodiments, the intraoral composition and the external composition may include about 8% to about 12% by weight of anti-inflammatory. In some embodiments, the amount of anti-inflammatory in the intraoral composition and the external composition may be about 8%, about 9%, about 10%, about 11%, about 12%, or about 13% by weight.

In some embodiments, the intraoral composition and the external composition may include about 0.5% to about 3% by weight of a muscle relaxant. In some embodiments, the amount of muscle relaxant in the intraoral composition and the external composition may be about 0.5%, about 1%, about 1.5%, about 2%, or about 2.5% by weight.

In some embodiments, the intraoral composition may include about 10% by weight of ketoprofen and about 2% by weight of cyclobenzaprine.

In some embodiments, the external composition may include about 10% by weight of ketoprofen and about 2% by weight of cyclobenzaprine.

In some embodiments, the external composition may include about 0.5% to about 4% by weight of an anesthetic. The some embodiments, the amount of anesthetic in the external composition may be 0.5%, about 1%, about 1.5%, about 2%, about 2.5%, about 3%, or about 4% by weight.

In some embodiments, the external composition may include about 10% by weight of ketoprofen, about 2% by weight of cyclobenzaprine, and about 1.5% by weight of lidocaine.

The compositions described above can be prepared by a pharmacist and stored in a topical cream pump or other application device. The topical cream pump provides a convenient and easy way for the patient to apply the compositions at-home.

In some embodiments, the external composition may be applied to the head or neck area, which may include skin overlaying a muscle of mastication or a tendon of a muscle of mastication. The muscles of mastication and tendons may include superficial masseters, deep masseter, anterior, middle, posterior temporalis muscles, medial pterygoid muscles, and the tendons of the lateral pterygoid muscles. In some embodiments, the external composition may be applied to the skin to treat secondary supportive musculature such as but not limited to sternocleidomastoid muscles, splenius capitis muscles, or trapezius muscles. In some embodiments, the external composition may be applied to an area overlaying a muscle of mastication that is sore or involuntarily contracting.

In some embodiments, the method may further include rubbing the external composition on the skin until it is visually undetectable. The external composition may be applied to areas covered or partially covered with hair.

The amount of the external composition to be applied may be an amount ranging from about 0.5 grams to about 4 grams. In some embodiments, the amount of the external composition to be applied ranges from about 1 gram to about 2 grams.

In some embodiments, the external composition may be applied to the head or neck area one, two, or three times per day. In some embodiments, the external composition may be applied to the head or neck area three times per day. The external composition may be applied when the patient awakens, between awakening and sleeping, and before the patient retires to bed.

In some embodiments, the intraoral area may include mucosa overlaying a muscle of mastication or a tendon of a muscle of mastication.

In some embodiments, the intraoral composition may be applied using an applicator. The applicator may comprise a pad or sponge. In some embodiments, a sponge may be loaded with the intraoral composition; thereby providing a controlled release of the composition from the sponge onto the target area. A non-limiting example of a sponge-type applicator is the 3M™ ESPE™ Disposable Mini-Sponge Applicators sold by 3M.

In some embodiments, the applicator may be held on the intraoral area for a duration of about 5 to about 60 seconds. In some embodiments, the applicator may be held in the target area for a duration of about 5 to about 30 seconds, about 5 to about 20 seconds, or about 5 to about 15 seconds. In some embodiments the applicator may be held in the intraoral area for a duration of about 10 seconds.

In some embodiments, the intraoral composition may be applied to an intraoral area 1, 2, 3, or 4 times per day. The amount of the intraoral composition to be applied may be an amount ranging from about 0.5 grams to about 4 grams. In some embodiments, the amount of the intraoral composition to be applied ranges from about 1 gram to about 2 grams.

In some embodiments, the intraoral composition may be applied after applying the external composition. The timing for applying the intraoral composition may depend upon pain ratings when palpating muscle trigger points. A common scale used by those treating pain is the Visual Analog Scale (VAS). The VAS is used to assess the patient's overall pain response and is measured on a scale from 1 to 10, with 10 being the most severe; however, a more useful and reliable scale for evaluating muscle trigger points associated with TMJD is 0 to 3, where one is slight, two is moderate, and three is severe.

In some embodiments, the intraoral composition may be applied when a qualitative pain measurement reaches a predetermined level. The qualitative pain measurement may be on a scale of 0 to 3, where three is the most severe. The various muscles of mastication and tendons may have different qualitative pain measurements. In some embodiments, the intraoral composition may be applied when the qualitative pain measurement of the primary muscles of mastication is reduced to a one and if the qualitative pain measurement of the lateral pterygoid muscles is a two or three. When it is determined that the intraoral composition can be applied, the external composition can be used in conjunction with the intraoral composition.

In other embodiments, a method for treating pain is disclosed. The method may include contacting skin with an external composition and contacting mucosal tissue in an oral cavity with an intraoral composition. The tissue in the intraoral cavity may be mucosal tissue. The external composition and the intraoral composition may an anti-inflammatory and a muscle relaxant, as described above.

The external and intraoral compositions can be used in combination with enteric medications, since no systemic effects have been observed. The compositions can be used on patients over the age of 65 and with patients with comorbidities, dizziness and vertigo, balance disorders, gaiting disorders, or orthopedic disabilities.

### EXAMPLES

Patients treated using the disclosed compositions and methods have reported many beneficial effects: decreased need for expensive and time consuming physical therapy, eliminated the need for invasive procedures such as dry needling or trigger point injections (lidocaine, steroids, or Botox), and increased patient response time during TMJD splint therapy. In addition, the compositions and methods have improved the overall treatment of the patient and has resulted in more predictable treatment with better outcomes.

### Example 1

A retrospective study was done on the clinical trials using an extraoral composition (about 2.5% by weight cyclobenzaprine; about 12% by weight ketoprofen; and 1.5% by weight lidocaine) and an intraoral composition (about 2% cyclobenzaprine and about 10% by weight ketoprofen) in the treatment of patients that were diagnosed with TMD's. Forty subjects between the ages of 15 and 94 were chosen. They were examined clinically and imaged using magnetic resonance imaging (MRI) when necessary. All were diagnosed with TMD. After diagnosis, the patients consented to treatment using orthopedic splint therapy, which is one of the most common treatment options for TMD's. All splints were mandibular, removable, and fabricated on a semi-adjustable articulator.

Patients were seen on a monthly basis for a duration of 9 to 12 months. During treatment, they were prescribed extraoral and intraoral compositions anywhere from the beginning to the end of splint therapy, depending upon the severity of their symptoms. The first 32 patients were treated with only the extraoral composition and 8 patients were treated with both the extraoral and the intraoral compositions.

All patients were evaluated at one month post-op (one month after starting use of the extraoral and intraoral compositions) and at the conclusion of the medical management. The patients were asked, “How much better or worse do you feel since starting us of the compositions?” at the one-month post-op appointment. They were also asked, “How much better or worse do you feel at the conclusion of using the compositions?”

The conclusion of using the extraoral and intraoral compositions coincided with the end of splint therapy. Patients were asked to give a percentage improvement or regression from a negative to a positive on a scale of 0 to 100.

At one-month post-op, there was an average of 40% improvement in the patients' overall pain response by adding use of the extraoral and intraoral compositions during splint therapy. At the conclusion of using the extraoral and intraoral compositions and splint therapy, the patients reported an average of 85% overall improvement to their pain response.

### Example 2

Another retrospective study was conducted with a control group that examined patients' pain response before using the extraoral and intraoral compositions in the treatment of patients diagnosed with TMD's. Thirty-nine subjects between the ages of 11 and 75 were chosen. As in the previous study, they were examined clinically and imaged using MRI's when appropriate. All subjects were diagnosed with TMD's. After diagnosis, the patients consented to treatment using orthopedic splint therapy. All splints were mandibular, removable and fabricated on a semi-adjustable articulator.

Once again, patients were seen on a monthly basis for a duration of 9 to 12 months. All patients were evaluated at one month post-op (one month after starting splint therapy) and at the conclusion of splint therapy. The patients were asked, “How much better or worse do you feel since starting splint therapy?” at the one month post-op appointment. They were also asked, “How much better or worse do you feel?” at the end of splint therapy. They were asked to give a percentage improvement, or regression from a negative to a positive on a scale of 0 to 100.

At the one-month post-op there was an average of 24% improvement in the patients' pain response when using splint therapy alone. At the conclusion of splint therapy, patients reported an overall improvement of their pain response by an average of 86%.

Ultimately, it was found there was a 16% greater improvement in how patients using the extraoral and intraoral compositions with splint therapy felt at the one-month post op than the control group using splint therapy alone.

When comparing the conclusion of splint therapy with use of the extraoral and intraoral compositions vs. splint therapy alone, the patients reported a similar overall improvement of their pain response (85% vs. 86%). While use of the extraoral and intraoral compositions will not affect the final result of splint therapy, when added as soon as clinically relevant to a patient's treatment protocol, one can expect an immediate decrease in the patient's perceived pain response compared to treatment with splint therapy alone.

### Example 3

When looking at both retrospective studies (Example 1 and 2) objectively, six areas of interest were targeted as follows: 1) trigger points (TP), 2) facial pain/jaw pain (FP/JP), 3) neck pain/shoulder pain (NP/SP), 4) range of motion (ROM), 5) capsulitis (caps), and 6) headaches (H).

‘Trigger points’ are characterized by local areas of firm, hypersensitive bands of skeletal muscle. ‘Facial pain, jaw pain, neck pain, and shoulder pain’ encompass many muscles or groups of muscles that are directly involved in TMJ and masticatory function, or are the source of myofacial referred pain. ‘Range of motion’ refers to the jaw opening of the mandible. ‘Capsulitis’ is an inflammation of the temporomandibular joint capsule. Finally, ‘headaches’ can be vascular, muscular, or both. The above six areas were measured at monthly examinations, but the results were tabulated at the end of treatment. The results are shown in Table 1.

TABLE 1

| | Experimental Group | |
|---|---|---|
| | (using the extraoral and | Control Group |
| | intraoral compositions) | (splint therapy only) |
| TP | 83% | 74% |
| FP/JP | 85% | 69% |
| NP/SP | 83% | 62% |
| ROM | 75% | 90% |
| Caps | 63% | 69% |
| H | 55% | 69% |

This comparative data demonstrated a significant improvement in trigger point/pain response, and in all muscle groups in the TMJ, masticatory muscle, and referral muscle groups seen in TMD's. By utilizing the extraoral and intraoral compositions, we were able to target all the muscles of mastication, versus solely targeting the extraoral muscles. This addition provided a significant improvement in decreased trigger point pain response and facial and jaw pain.

From the tables above, range of motion, capsulitis, and headache did not show improvement. Range of motion is a broad and complex process that involves the fibrocartilage discs of the temporomandibular joints. Considering capsulitis, inflammation of the temporomandibular joint capsule can involve the underlying synovium of the temporomandibular joint, which provides synovial fluid and become inflamed as well (synovitis). Finally, headaches have a vascular component, which can cause vasodilation of the endothelium of the arteries, causing headaches.

Another area of interest from both studies involved the use of oral medications during the treatment of TMD's. The oral medication types used were: 1. skeletal muscle relaxants, 2. NSAID's, 3. tryptophans, and 4. opioids (narcotic analgesics). Flexeril and Amrix were the skeletal muscle relaxants used, and the NSAID's used were Celebrex and Motrin. The tryptophans used were Relpax and Zecuity, and the opioid used in one patient was Vicoprofen. In the experimental group there was an 11% decrease in the use of oral medications compared to the control group. This is significant as one can decrease the side effects of these medications and their potential for addiction. Also, using the extraoral and intraoral compositions topically does not add systemically to the patient's current medications.

The final area of inquiry for both retrospective studies was examining each patient's medical history. We looked at co-morbidity, age of the patient, and disabilities. Co-morbidities affecting the region of TMD's were: cervical spine, fibromyalgia, Ehler's Danlos, oral dystonia, Lyme disease, neuropathic pain, chronic pain, and chronic fatigue. The age range of patients was between 11 and 94. Disabilities included: orthopedic injuries, balance disorders, and depression.

When comparing the two groups regarding co-morbidities, all of the above areas were significantly affected when using the extraoral and intraoral compositions, as they were in the targeted treatment areas of TMD's. Additionally, the extraoral and intraoral compositions can be applied at any age. Most oral medications in the types outlined above come with limitations over the age of 65. And finally, patients with disabilities can safely use the extraoral and intraoral compositions without affecting their equilibrium, gait, or mood.

Conventional treatment of TMD commonly involves the use of splint therapy, but oftentimes includes the use of oral medications. Additional treatments may include: physical therapy, chiropractic, acupuncture, trigger point injections, Botox, and psychological management. All of these are time consuming and expensive. The use of a compounded transdermal medication (extraoral and intraoral compositions) in the treatment of TMD can specifically target the areas of the TMD without the medical challenges associated with oral medications. Convenience adds another benefit, as the use of additional treatments can almost entirely be eliminated, saving the patient time and money. Therefore, by adding the extraoral and intraoral compositions to a TMD treatment protocol like splint therapy, one can simultaneously achieve a decreased patient response to pain and an increased effectiveness to therapy.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a device” is intended to include “at least one device” or “one or more devices.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of augmenting temporomandibular disorder (TMD) splint therapy, comprising:
inserting an orthopedic, mandibular splint to a patient suffering from a temporomandibular joint disorder into the patient's mouth, where the splint is mandibular.
applying an intraoral composition to a lateral pterygoid space using an applicator;
applying an external composition to a head or neck area;
wherein the external composition and the intraoral composition comprise an anti-inflammatory and a muscle relaxant;

2. The method of claim 1, wherein the anti-inflammatory is a non-steroidal anti-inflammatory drug.

3. The method of claim 1, wherein the anti-inflammatory in the external composition and the intraoral composition is independently selected from the group consisting of celecoxib, diclofenac, diflunisal, etodolac, ibuprofen, indomethacin, ketoprofen, ketorolac, meloxicam, nabumetone, naproxen, oxaprozin, piroxicam, salsalate, sulindac, tolmetin, and any combination thereof.

4. The method of claim 1, wherein the anti-inflammatory is ketoprofen.

5. The method of claim 1, wherein the muscle relaxant in the external composition and the intraoral composition is independently selected from the group consisting of carisoprodol, chlorzoxazone, cyclobenzaprine, metaxalone, methocarbamol, orphenadrine, and any combination thereof.

6. The method of claim 1, wherein the muscle relaxant is cyclobenzaprine.

7. The method of claim 1, wherein the external composition further comprises an anesthetic.

8. The method of claim 1, wherein the external composition further comprises lidocaine.

9. The method of claim 1, wherein the external composition further comprises a carrier.

10. The method of claim 1, wherein the external composition further comprises an oil-in-water emulsion.

11. The method of claim 1, wherein the intraoral composition further comprises a plasticized ointment.

12. The method of claim 1, wherein the applicator is a sponge providing a controlled release of the intraoral composition.

13. The method of claim 1, wherein the head or neck area comprises skin overlaying a muscle of mastication or a tendon of the muscle of mastication.

14. The method of claim 1, wherein the intraoral area comprises mucosa overlaying a muscle of mastication or a tendon of the muscle of mastication.

15. The method of claim 1, wherein the intraoral composition is applied after applying the external composition.

16. The method of claim 1, wherein the intraoral composition is applied when a qualitative pain measurement reaches a predetermined level.

17. The method of claim 1, wherein the intraoral composition and the external composition comprise about 5% to about 15% by weight of ketoprofen and about 0.5% to about 2.5% by weight of cyclobenzaprine.
